(19) United States
(12) Patent Application Publication
(10) Pub. No.: US 2008/0161560 A1
(43) Pub. Date: Jul. 3, 2008

(54) Process for Preparation of Calcium Salt of Rosuvastatin

(76) Inventors: Pandurang Balwant Deshpande, Mumbai (IN); Arul Ramakrishnan, Mumbai (IN); Balkrishna Shrigadi Nilesh, Mumbai (IN); Mukunda Bahul Sandeep, Mumbai (IN)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 11/816,155

(22) PCT Filed: Aug. 9, 2005
(86) PCT No.: PCT/IN2005/000265

§ 371 Date: Aug. 13, 2007

(30) Foreign Application Priority Data
Apr. 4, 2005 (IN) 425/MUM/2005

(51) Int. Cl. C07D 239/42

(52) U.S. Cl. 544/297

(57) ABSTRACT

The invention relates to commercially viable process for the preparation of Rosuvastatin by an early introduction of correct absolute stereochemistry at C-5 (S) of Rosuvastatin side chain followed by regioselective chain extension using novel side chain building blocks and less expensive reagents. It is yet another object of the invention is to provide novel intermediates that may be used for the preparation of Calcium salt of Rosuvastatin.Formula (I).

### FIELD OF THE INVENTION

The present invention relates to a process for the preparation of Rosuvastatin, a promising HMG-CoA reductase inhibitor, to process steps and novel intermediates.

### BACKGROUND OF THE INVENTION

HMG-CoA reductase inhibitors (also called β-hydroxy-β-methylglutaryl-co-enzyme-A reductase inhibitors and also called statins) are understood to be those active agents, which may be preferably used to lower the low-density lipoprotein (LDL) particle concentration in the blood stream of patients at risk for cardiovascular disease and thus used for the prevention or treatment of hypercholesterolemia, hyperlipoproteinemia and artheriosclerosis. A high risk level of LDL in the bloodstream has been linked to the formation of coronary lesions that obstruct the flow of blood and can rupture and promote thrombosis.

Rosuvastatin, an antihyperchlolesterolemic drug used in the treatment of atherosclerosis is chemically (E)-7-[4-(4-flurophenyl)-6-isopropyl-2-[methyl(methylsulfonyl)amino]pyrimidin-5-yl](3R,5S)-3,5-dihydroxyhept-6-enoic acid calcium (2:1) salt having the structural formula I.

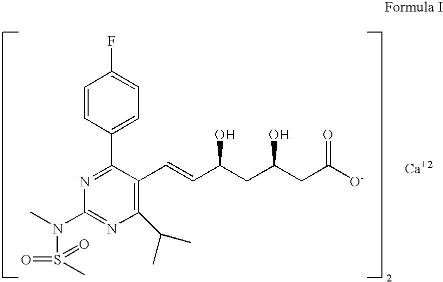

The prior art has been elaborated in a co-pending application number 325/MUM/2005 filed in Mumbai, India.

The above mentioned co-pending application discloses a commercially viable method of preparation for Rosuvastatin via novel intermediates.

In continuation of our work in this aspect, in this present invention relates another method of preparation of Rosuvastatin via novel intermediates and less expensive reagents by an early introduction of the correct absolute stereochemistry at C-5 (S) of side chain followed by regioselective chain extension using yet another side chain building blocks. The invention also relates to novel intermediates.

### DETAILED DESCRIPTION OF THE INVENTION

The present invention concerns a process for the preparation of rosuvastatin comprising

a) reacting a compound of formula (II)

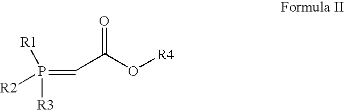

wherein, R1, R2, R3 are substituted or unsubstituted phenyl and R4 is an aliphatic residue selected from C1-C4 alkyl;
with a compound of formula R—CH(═O) (Formula III) wherein R represents the following cyclic structure (formula IV) to obtain a compound of formula (V);

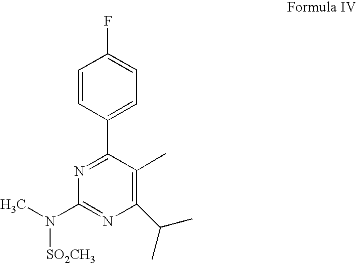

b). hydrolysing a compound of formula (V) to obtain a compound of formula (VI);

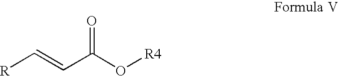

c). treating a compound of formula (VI)

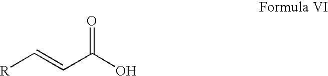

with an acid activating group and subsequently with a compound of formula VII that introduces the radical of formula —CH₂—COOR5 to obtain a compound of formula VIII

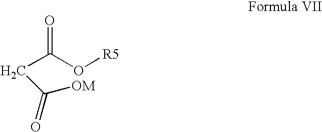

wherein, R5 represents C1-C4 alkyl; M is an alkali metal;
or in another variant of process, converting the compound of formula (VI) to its acid halide of formula (IX)

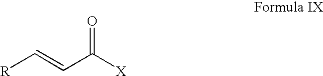

wherein, X represents a halogen
and treating a resulting compound of formula (IX) with a compound of formula (X) to obtain a compound of formula (VIII);

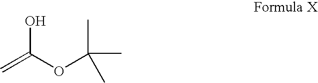

or in another variant of process, treating the compound of formula (IX) with a compound of formula (VII) to obtain a compound of formula (VII);

d). reducing a compound of formula (VIII) to obtain a compound of formula XI;

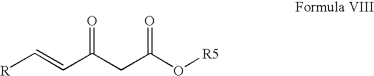

e). hydrolyzing a compound of formula (XI) to obtain a compound of formula XII

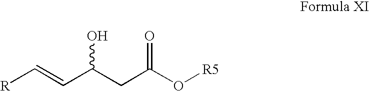

f). resolving the resulting racemic compound of formula (XII), first converting the racemic compound to its diastereomeric salt using the (+) or (−) enantiomeric amine of the formula (XIII) and separating the mixture of diastereomeric salt into the individual diastereomers by chromatography or crystallization and then neutralizing the diastereomeric salt to give the enantiomerically pure product.

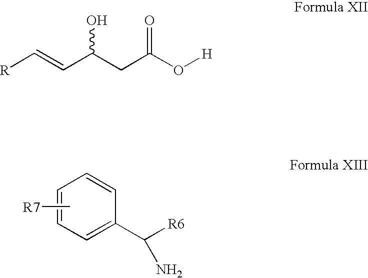

wherein, R6 represent C1-C4-alkyl which is optionally substituted by hydroxyl; R7 represent hydrogen, halogen, C1-C4 alkyl or C1-C4 alkoxy; and

g). treating the resulting compound of formula (XIV)

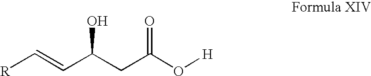

with an acid activating group and subsequently with a compound of formula (VII) that introduces the radical of formula —CH₂—COOR5 to obtain a compound of formula (XV) or in another variant of process, esterifing a compound of formula (XIV)

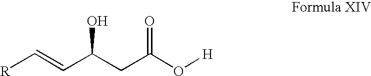

and condensing the resulting compound of formula (XVI)

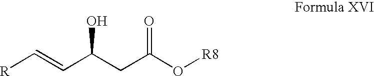

wherein R8 is an aliphatic residue selected from C1-C4 alkyl
with a compound of formula (X) to obtain a compound of formula (XV)

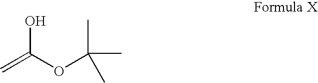

h). reducing a compound of formula (XV) to obtain a compound of formula XVII

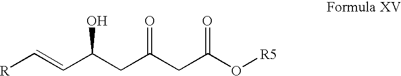

i). hydrolyzing a compound of formula (XVII) and converting into a salt of formula I thereof

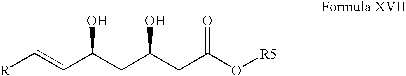

wherein R and R5 have the meanings as defined.

In reaction step (a), the reaction of a compound of formula (II) with a compound of formula (III) is carried out in a suitable inert solvent, preferably toluene, and in a temperature range from 60° C., to the boiling point of the solvent, preferably at the boiling point of the solvent.

The saponification (step b) is carried out by treating the ester of formula (V) with a strong base, such as an alkali metal hydroxide, preferably NaOH or KOH, in aqueous aliphatic alcohol as solvent, preferably aqueous methanol, and in a temperature range from 25° C. to boiling point of solvent, preferably between 25° C. to 35° C. and acidifying the resulting reaction mixture.

Formation of compound of formula (VIII) (step c) is carried out by treating the compound of formula (VI) with an acid activating group, especially preferred one is the use of 1,1-carbonyldiimidazole and condensing the resulting compound with alkali metal salt of manoalkyl malonate (formula VII), preferably potassium monomethylmalonate or potassium monoethylmalonate, in the presence of magnesium chloride, in an inert solvent, preferably tetrahydrofuran, at temperature between 0-40° C., preferably at 0-35° C.

In another variant of process to prepare compound of formula (VIII) is carried out by converting a compound of formula (VI) to a compound of formula (IX) in an inert solvent, preferably dichloromethane, and in temperature range from 0° C. to boiling point of the solvent preferably between 0° C. to 28° C. using oxalyl chloride or thionyl chloride and subsequent treatment of a resulting of formula of (IX) with a compound of formula (X) in the presence of a suitable base and in a suitable inert solvent, especially tetrahydrofuran, and in a temperature range from −78° C., to the boiling point of the solvent, preferably at −78 to room temperature.

A suitable base is selected from an alkane alkali metal in presence of diisopropylamine, alkali alkylsilazanes like LiHMDS or NaHMDS. Especially preferred is the use of n-butyllithium in the presence of diisopropylamine.

In another variant of process to prepare compound of formula (VIII) is carried out by condensing a compound of formula (IX) with an alkali metal salt of manoalkyl malonate (Formula VII), preferably potassium monomethylmalonate or potassium monoethylmalonate, in the presence of magnesium chloride, in an inert solvent, preferably tetrahydrofuran, at temperature between 0-40° C., preferably at 0-35° C.

The reduction of compound of formula (VIII), is carried out in a mixture of an inert solvent, such as an ether, preferably tetrahydrofuran and lower alkanol, preferably methanol, in the ratio of 4:1 volume/volume, and at temperature range from −78° C. to 0° C., preferably at −65° C. to 0C.

A preferred reduction agent is a hydride, for example, an alkalimetal borohydride especially sodium borohydride.

The saponification step e) is carried out by treating the ester of formula (XI) with a strong base, such as an alkali metal hydroxide, preferably NaOH or KOH, in aqueous aliphatic alcohol as solvent, preferably aqueous methanol, and in a temperature range from 25° C. to boiling point of solvent, preferably between 25° C. to 30° C. and acidifying the resulting reaction mixture.

The resolution of racemate of compound of formula (XII) (step f) in to optically pure antipodes is carried out by means of known methods for the separation of entiomers, for example by means of preparative chromatography using chiral supports (HPLC) or by crystallization using optically pure precipitating agents, for example (+) or (−) phenylalkylamine or substituted phenylalkylamine, preferably (R)-1-phenylethylamine in alcoholic solvents such as lower alkanol, preferably ethanol and recrystallising from a mixture of ketonic solvent and lower alkanol, preferably mixture of acetone and methanol followed by neutralization.

Formation of compound of formula (XV) is carried out by treating the compound of formula (XIV) with an acid activating group especially preferred one is the use of 1,1-carbonyldiimidazole and condensing the resulting compound, with alkali metal salt of manoalkyl malonate (Formula VII), preferably potassium monomethylmalonate or potassium monoethylmalonate, in presence of magnesium chloride, in an inert solvent, preferred one is tetrahydrofuran, at temperature between 0-40° C. preferably at 0-35° C. In another variant of process to prepare compound of formula (XV) is carried out by converting compound of formula XIV to a compound of formula XVI by esterification and condensing the resulting compound of formula (XVI) with a compound of formula (X).

Esterification of compound of formula XIV is carried out, in lower alcoholic solvent, especially C1-C3 alkanol, preferably methanol, in presence of acidic catalyst like inorganic acids or p-toluensulphonic acid or acidic resins, and in a temperature range from 0° C. to boiling point of solvent, preferably between 0° C. to 30° C.

Condensation step is carried out in the presence of a suitable base and in a suitable inert solvent, especially tetrahydrofuran, and in a temperature range from −78° C. to the boiling point of the solvent, preferably at room temperature. A suitable base is selected from an alkane alkalimetal in the presence of diisopropylamine, alkali alkylsilazanes like LiHMDS or NaHMDS. Preferred one is the use of n-butyllithium in the presence of diisopropylamine.

The reduction of compound of formula XV (step h), is carried out in a mixture of an inert solvent, preferably tetrahydrofuran and a lower alkanol, preferably methanol, in the ratio of 4:1 volume/volume, and at temperatures from −78° C. to 0° C., preferably at −78° C. to −70° C. To split the corresponding boronic ester the reaction mixture is then treated with methanol, and in a temperature range from 0° C. to the boiling point of solvent, preferably in range of 0° C. to 40° C.

A preferred reduction agent is an alkalimetal borohydride in the presence of a di-C1-C7-alkyl-C1-C4 alkoxy-borane, especially sodium borohydride in the presence of diethylmethoxyborane.

The isolation of compound of formula I (step i), is carried out by saponification of a Compound of formula XVII using a base, such as an alkali metal hydroxide, preferably NaOH and followed by treatment with aqueous calcium chloride solution.

The present invention also relates to a novel compound of formula VI or its acid chloride and process of making it.

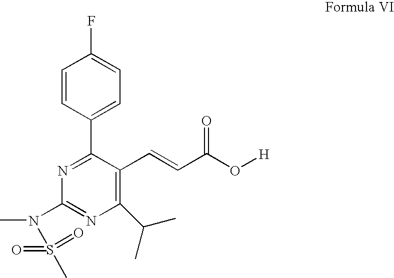

The starting material of formula III may be prepared, for example, as described in Bioorganic & Medicinal Chemistry 1997, 437.

In the following examples, the preferred embodiments of the present invention are described only by way of illustrating the process of the invention. However, these are not intended to limit the scope of the present invention in any way.

### EXAMPLE 1

### Preparation of Ethyl (2E)-3-{4-(4-flurophenyl)-6-isopropyl-2-[methyl(methylsulfonyl)amino]pyrimidin-5-yl}acrylate

To a solution of N-[4-(4-flurophenyl)-5-formyl-6-isopropylpyrimidin-2-yl]-N-methylmethylsulfonamide (55 g; 156 mmol) in 700 ml of toluene, 60.2 g of (carbethoxymethylene)triphenylphosphorane (172 mmol) was added at 25-29° C. The reaction mixture was refluxed for 6 hours. After completion of reaction (TLC; disappearance of starting material), reaction mixture was cooled between 25-28° C. and 500 ml of n-hexane was added and stirrer for 15 minutes. The separated solid was removed by filtration and the filtrate was distilled under reduced pressure to remove the solvents. The oily mass obtained after removal of solvents was purified through silica gel column to obtain ethyl (2E)-3-{4-(4-flurophenyl)-6-isopropyl-2-[methyl(methylsulfonyl) amino]pyrimidin-5-yl}acrylate as a solid.

¹H NMR (400 MHz, CDCl₃): 1.27-1.3 (9H, m, —CH(CH₃)₂, —CH₂CH₃), 3.33-3.4 (1H, m, —CH(CH₃)₂, 3.49 (3H, s, —NCH₃), 3.55 (3H, S, —SO₂CH₃), 4.19 (2H, q, —OCH₂CF13), 5.81 (1H, d, J=16.10) 5.81 (1H, d, C═CHCOOCH₂), 7.10 (2R, t, Ar—H), 7.59 (2w, dd, Ar—H), 7.68 (1H, d, J=16.10, —CH═CHCOOCH₂). ¹³C NMR (400 MHz, CDCl₃): 14.32, 21.97, 30.01, 32.29, 42.44, 60.76, 115.45, 115.67, 118.81, 125.71, 132.04, 132.73, 133.67, 133.71, 139.17, 157.97, 162.51, 164.33, 165.01, 165.50, 175.15

### EXAMPLE 2

### Preparation of (2E)-3-{4-(4-flurophenyl)-6-isopropyl-2-[methyl(methyl sulfonyl)amino]pyrimidin-5-yl}acrylic Acid

A solution of ethyl (2E)-3-{4-(4-flurophenyl)-6-isopropyl-2-[methyl(methyl sulfonyl)amino]pyrimidin-5yl}acrylate 20 g (47.5 mmol) in methanol (200 ml). To this solution, NaOH (2.09 g; 52.25 mmol)) in 50 ml of water was added in drop wise over the period of approximately 15 minutes at temperature between 25° C. to 29° C. After stirring at this temperature for further 8 hours, to the reaction mixture 200 ml of tert-butyl methyl ether was added followed by 50 ml of water. The aqueous layer was separated and the organic layer was washed with 50 ml of water. The aqueous layers were combined and the pH was adjusted to approximately 3-4 by acidification and extracted twice to 200 ml of dichloromethane. The combined organic layers were washed with 100 ml saturated NaCl solution, dried over anhydrous Na₂SO₄ and filtered. The filtrate obtained was evaporate to dryness under vacuum to obtain (2E)-3-{4-(4-flurophenyl)-6-isopropyl-2-[methyl (methylsulfonyl)amino]pyrimidin-5-yl}acrylic acid as a white solid. The structure of the product has been conformed by NMR, Mass.

¹H NMR (400 MHz, CDCl₃) δ=1.23 (6H, d, —CH(CH₃)₂); 3.33-3.4 (1H, m, —CH(CH₃)₂); 3.45 (3H, s, —NCH₃); 3.52 (3H, S, —SO₂CH₃); 5.8 (1H, d, J=16.34, ═CH—COOH); 7.06 (2H, t, Ar—H), 7.53 (2H, dd, Ar—H); 7.75 (1H, d, J=16.10, —CH═CH—COOH); 9.8 (1H, br. s, COOH).

### EXAMPLE 3

### Preparation of Methyl (4E)-5-{4-(4-flurophenyl)-6-isopropyl-2-[methyl (methylsulfonyl)amino]pyrimidin-5-yl}-3-oxo-4-pentenoate

Method 1:

To a solution of (2E)-3-{4-(4-flurophenyl)-6-isopropyl-2-[methyl(methylsulfonyl) amino]pyrimidin-5-yl}acrylic acid (2.0 g; 5.06 mmol) in 8 ml of tetrahydrofuran (THF), 1,1-carbonyldiimidazole (0.98 g: 6.07 mmol) was added in portions over a period of 5 minutes and stirred between 25° C. and 29° C. under nitrogen atmosphere. After stirring for 2 hours this solution was added to a preformed mixture of monomethyl malonate potassium salt (0.79 g; 5.06 mmol), magnesium chloride (0.482 g, 5.06 mmol and triethylamine which was stirred for further 2 hours at 25-28° C. The resulted reaction mixture was stirred for 24 hours at 35° C. The reaction mixture was cooled to approximately to 27° C. and filtered. The residue was washed twice with 25 ml of THF and combined with the filtrate. The combined filtrate was concentrated under vacuum and the residue obtained was dissolved in 60 ml of ethyl acetate, washed with 30 ml of 1 N hydrochloric acid, thrice with 40 ml of saturated NaHCO₃ followed by saturated NaCl solution, dried over anhydrous Na₂SO₄. The filtrate obtained after the filtration was concentrated under reduced pressure to obtain methyl (4E)-5-{4-(4-flurophenyl)-6-isopropyl-2-[methyl(methylsulfonyl)amino]pyrimidin-5-yl}-3-oxo-4-pentenoate as a yellow colored solid.

¹H NMR (400 MHz, CDCl₃): 1.26 (6H, d, —CH(CH₃)₃); 3.3-3.38 (1H, m, —CH(CH₃)₃); 3.49 (3H, s, —NCH₃); 3.55 (3H, s, —SO₂CH₃); 3.7 (3H, s, OCH₃); 4.94 (1H, s); 5.72 (1H, d, J=15.85, Ar—CH═CH—); 7.08 (2H, t, Ar—H); 7.42 (1H, d, J=15.85′ Ar—CH═CH—); 7.61 (2H, dd, Ar—H), 11.79 (1H, s, enol-OH).

Method 2:

a). Preparation of (2E)-3-{4-(4-flurophenyl)-6-isopropyl-2-[methyl(methylsulfonyl) amino]pyrimidin-5-yl}-2-propenoyl chloride

To a solution of (2E)-3-{4-(4-flurophenyl)-6-isopropyl-2-[methyl(methylsulfonyl) amino]pyrimidin-5-yl}acrylic acid (2.0 g; 5.06 mmol) in 20 ml of dichloromethane oxalyl chloride (0.77 g; 6.07 mmol) was added in drop wise over a period of 5 minutes at 0-5° C. with stirring under nitrogen atmosphere. The reaction mixture was allowed to warm up and stirred at 25° C. to 29° C. After stirring for 2 hours, the reaction mixture was concentrated under reduced pressure and swapped trice with 20 ml dichloromethane to obtained (2E)-3-{4-(4-flurophenyl)-6-isopropyl-2-[methyl(methylsulfonyl)amino]pyrimidin-5-yl}-2-propenoyl chloride as a solid.

b). Preparation of methyl (4E)-5-{4-(4-flurophenyl)-6-isopropyl-2-[methyl (methylsulfonyl)amino]pyrimidin-5-yl}-3-oxo-4-pentenoate

To a solution of monomethyl malonate potassium salt (0.79 g; 5.06 mmol), magnesium chloride (0.482 g; 5.06 mmol) was added followed by triethylamine (0.51 g) and the suspension was stirred for 2 hours at 25-28° C. To this, solution of (2E)-3-{4-(4-flurophenyl)-6-isopropyl-2-[methyl(methylsulfonyl)amino]pyrimidin-5-yl}-2-propenoyl chloride (2.06 g, 5.0 mmol) in 10 ml of THF was added and stirred for 24 hours at 30-35° C. The reaction mixture was cooled, filtered and then residue was washed twice with 25 ml of THF. The filtrate was concentrated by evaporation, taken up in 60 ml of ethyl acetate, washed with 30 ml of 1N hydrochloric acid, thrice with 40 ml of saturated NaHCO₃ solution followed by saturated NaCl solution and dried over anhydrous Na₂SO₄. The filtrate after filtration was concentrated under reduced pressure to obtain methyl (4E)-5-{4-(4-flurophenyl)-6-isopropyl-2-[methyl(methylsulfonyl)amino]pyrimidin-5-yl}-3-oxo-4-pentenoate as yellow colored solid.

### EXAMPLE 4

### Preparation of racemic methyl (4E)-5-{4-(4-flurophenyl)-6-isopropyl-2-[methyl(methylsulfonyl)amino]pyrimidin-5-yl}-3-hydroxy-4-pentenoate

To a solution of methyl (4E)-5-{4-(4-flurophenyl)-6-isopropyl-2-[methyl(methyl sulfonyl)amino]pyrimidin-5-yl}-3-oxo-4-pentenoate (1.5 g; 3.325 mmol) in 15 ml of THF/methanol (4:1) was cooled to −65° C. under nitrogen atmosphere with stirring. To this stirred solution, NaBH₄ (0.154 g; 3.99 mmol) was added in portion and solution was stirred for further 1-2 hours at −65° C. To this 1 ml of acetic acid in 15 ml water followed by 15 ml ethyl acetate was added and stirred for 5 min. The layers were separated and aqueous layer was extracted twice with 30 ml of ethyl acetate. The combined organic phases are washed twice with 30 ml saturated NaHCO₃ solution and then with 30 ml saturated NaCl solution, dried over anhydrous Na₂SO₄, filtered. The filtrate was concentrated under reduced pressure to obtained methyl (4E)-5-{4-(4-flurophenyl)-6-isopropyl-2-[methyl(methylsulfonyl)amino]pyrimidin-5-yl}-3-hydroxy-4-pentenoate as solid.

¹H NMR (400 MHz, CDCl₃): 1.21 (6H, d, —CH(CH₃)₃), 2.3-2.5 (2H, m, CH₂—COO); 3.27-3.34 (1H, m, —CH(CH₃)₃), 3.49 (3H, s, —NCH₃), 3.54 (3H, s, —SO₂CH₃); 3.68 (3H, s, —OCH₃); 4.52-5.56 (1H, m, >CH—OH); 5.45 (1H, d, J=16.10, ═CHCOO), 6.64 (1H, d, J=16.10, CH═CHCOO); 7.07 (2H, t, Ar—H), 7.6 (2H, dd, Ar—H).

### EXAMPLE 5

### Preparation of racemic (4E)-5-{4-(4-flurophenyl)-6-isopropyl-2-[methyl (methylsulfonyl)amino]pyrimidin-5-yl}-3-hydroxy-4-pentenoic acid

To a stirred solution of racemic methyl (4E)-5-{4-(4-flurophenyl)-6-isopropyl-2-[methyl(methylsulfonyl)amino]pyrimidin-5-yl}-3-hydroxy-4-pentenoate (12 g; 26.6 mmol) in 120 ml of methanol, a solution of aqueous sodium hydroxide (1.17 g; 29.3 mmol in 25 ml water) was added slowly at temperature between 27-29° C. and stirred for further 1-2 hours. After completion of reaction (TLC; disappearance of starting material), 25 ml of water and 120 ml tert-butyl methyl ether were added. The organic layer was separated and washed with 25 ml water. The aqueous layers were combined and the pH was adjusted to approximately 3-4 by acidification and extracted twice to 100 ml of dichloromethane. The combined organic layers were washed with 50 ml saturated NaCl solution, dried over anhydrous Na₂SO₄, filtered. The filtrate obtained was evaporated to dryness under vacuum to obtain racemic (4E)-5-{4-(4-flurophenyl)-6-isopropyl-2-[methyl(methylsulfonyl)amino]pyrimidin-5-yl}-3-hydroxy-4-pentenoic acid as off-white solid. The structure of the product has been conformed by NMR.

¹H NMR (400 MHz, CDCl₃): 1.3 (6H, d, —CH(CH₃)₃), 3.34-3.41 (1H, m, —CH(CH₃)₃), 3.47 (3H, s, —NCH₃), 3.56 (3H, s, —SO₂CH₃), 5.85 (1H, d, J=16.34, ═CHCOOH), 7.12 (2H, t, J=8.29, Ar—H), 7.59 (2H, dd, J=8.05, 5.51, Ar—H), 7.8 (1H, d, J=16.34, —CH═CHCOOH), 10.79 (1H, br., —COOH). ¹³C NMR (400 MHz, CDCl₃): 21.96, 32.37, 33.03, 42.45, 115.56, 115.77, 118.39, 124.88, 132.04, 132.13, 133.60, 133.59, 141.59, 158.13, 162.57, 164.60, 165.07, 170.91, 175.23.

### EXAMPLE 6

### Preparation of (4E)-5-{4-(4-flurophenyl)-6-isopropyl-2-[methyl (methylsulfonyl)amino]pyrimidin-5-yl}(3S)-3-hydroxy-4-pentenoic Acid

To a solution of racemic (4E)-5-{4-(4-flurophenyl)-6-isopropyl-2-[methyl(methyl sulfonyl)amino]pyrimidin-5-yl}-3-hydroxy-4-pentenoic acid in ethanol (R)-1-phenyl ethylamine was added at 25-29° C. The reaction mixture was cooled to 0° C. and stirred for another 3 hours. The solid precipitated was filtered and washed with tert-butyl methyl ether, dried under vacuum. The solid obtained after drying was recrystallised from 5 volumes of methanol-acetone mixture (1:4 ratio by v/v) to get (R)-1-phenylethylamine salt of (4E)-5-{4-(4-flurophenyl)-6-isopropyl-2-[methyl(methylsulfonyl)amino]pyrimidin-5-yl}(3S)-3-hydroxy-4-pentenoic acid.

The crystallised salt was taken in methanol and treated with aqueous sodium hydroxide solution at 25-28° C. with stirring. After stirring for 1 hour, water was added followed by tert-butyl methyl ether. The organic layer was separated and the aqueous layer was acidified (pH of 3-4) and extracted with dichloromethane. After removal of solvent under vacuum, (4E)-5-{4-(4-flurophenyl)-6-isopropyl-2-[methyl(methylsulfonyl)amino]pyrimidin-5-yl}(3S)-3-hydroxy-4-pentenoic acid was obtained as a solid.

¹H NMR (400 MHz, CDCl₃): 1.3 (6H, d, —CH(CH₃)₃), 3.34-3.41 (1H, m, —CH(CH₃)₃), 3.47 (3H, s, —NCH₃), 3.56 (3H, s, —SO₂CH₃), 5.85 (1H, d, J=16.34, ═CHCOOH), 7.12 (2H, t, J=8.29, Ar—H), 7.59 (2H, dd, J=8.05, 5.51, Ar—H), 7.8 (1H, d, J=16.34, —CH═CHCOOH), 10.79 (1H, br s, —COOH). ¹³C NMR (400 MHz, CDCl₃): 21.96, 32.37, 33.03, 42.45, 115.56, 115.77, 118.39 124.88. 132.04, 132.13, 133.60, 133.59, 141.59, 158.13, 162.57, 164.60, 165.07, 170.91, 175.23.

### EXAMPLE 7

### Preparation of Methyl (6E)-7-{4-(4-flurophenyl)-6-isopropyl-2-[methyl (methylsulfonyl)amino]pyrimidin-5-yl}(5S)-5-hydroxy-3-oxo-6-heptenoate

To a solution of (4E)-5-{4-(4-flurophenyl)-6-isopropyl-2-[methyl(methylsulfonyl) amino]pyrimidin-5-yl}(3S)-3-hydroxy-4-pentenoic acid (2.0 g; 4.57 mmol) in 8 ml of THF, 1,1-carbonyldiimidazole (0.885 g, 5.48 mmol) was added in portions over a period of 5 minutes and stirred between 25° C. and 29° C. under nitrogen atmosphere. After stirring for 2 hours, this solution was added to a preformed mixture of monomethyl malonate potassium salt (0.71 g; 4.57 mmol), magnesium chloride (0.435 g, 4.57 mmol) and triethylamine (0.46 g; 4.57 mmol) which was stirred for further 2 hours at 25-28° C. The resulted reaction mixture was stirred for 24 hours at 30-35° C. The reaction mixture was cooled and filtered and then residue is washed twice with 25 ml of THF. The filtrate was concentrated by evaporation, taken up in 60 ml of ethyl acetate, washed with 30 ml of 1N hydrochloric acid, thrice with 40 ml of saturated NaHCO₃ solution followed by saturated NaCl solution, dried over anhydrous Na₂SO₄. The filtrate obtained after filtration was concentrated under reduced pressure to obtain yellow colored oily mass, which was purified through column to obtain methyl (6E)-7-{4-(4-fluorphenyl)-6-isopropyl-2-[methyl(methylsulfonyl)amino]pyrimidin-5-yl}(5S)-5-hydroxy-3-oxo-6-heptenoate.

¹H NMR (400 MHz, CDCl₃): δ=1.2 (6H, d, —CH(CH₃)₂); 2.6 (2H, d, (OH)CH—CH₂—C(O)); 3.3 (1H, m, —CH(CH₃)₂); 3.43 (2H, s, (O)C—CH₂—COO—); 3.47 (3H, s —NCH₃); 3.52 (3H, s, —SO₂CH₃); 3.7, (3H, s, —COOCH₃); 4.61 (1H, m, >CH—OH); 5.4 (1H, dd, J=16, ═CH—CH(OH); 6.6 (1H, d, 15.85, Ar—CH═CH); 7.1 (2H, t, Ar—H0; 7.6 (2H, dd, Ar—H).

### EXAMPLE 8

### Preparation of methyl (6E)-7-{4-(4-flurophenyl)-6-isopropyl-2-[methyl(methylsulfonyl)amino]pyrimidin-5-yl}(3R,5S)-3,5-dihydroxyhept-6-enoate

Methyl (6E)-7-{4-(4-flurophenyl)-6-isopropyl-2-[methyl(methylsulfonyl)amino]pyrimidin-5-yl}(5S)-5-hydroxy-3-oxo-6-heptenoate (1 g; 2.03 mmol) was taken in 10 ml dry THF/methanol (4:1) and cooled to −78° C. under nitrogen atmosphere. To this stirred solution, diethylmethoxyborane (1 M in THF; 0.223 g; 2.23 mmol) was added drop wise over a period of ˜5 minutes. After stirring at that temperature for 30 minutes, NaBH₄ (0.076 g; 2.23 mmol) was added at −78° C. and stirred at −78° C. for 3-4 hours. To this reaction mixture, 1 ml of acetic acid was added in drop wise followed by 10 ml of ethyl acetate and 10 ml of water. After stirring for 10 minutes at −78° C., the reaction mixture allowed reach 25-28° C. The layers were separated and the aqueous layer was extracted twice with 30 ml of ethyl acetate. The combined organic layers were washed twice with 30 ml saturated NaHCO₃ solution and then with saturated NaCl solution, dried over anhydrous Na₂SO₄. The reaction mixture was filtered and the solvents were removed by distillation under vacuum. The oily product thus obtained was swapped thrice with 30 ml of methanol to remove borate complex and concentrated to obtained oily mass, which after column purified provided methyl (6E)-7-{4-(4-flurophenyl)-6-isopropyl-2-[methyl(methylsulfonyl)amino]pyrimidin-5-yl}(3R,5S)-3,5-dihydroxyhept-6-enoate.

¹H NMR (400 MHz, CDCl₃): δ=1.2 (6H, d, —CH(CH₃)₂); 1.39-1.56 (2H, m, >CH₂); 2.4 (2H, CH—CH₂—COO); 3.3 (1H, m, —CH(CH₃)₂); 3.48 (3H, s —NCH₃); 3.53 (3H, s, SO₂CH₃); 3.7, (3H, s, —COOCH₃); 4.16 (1H, m, >CH—OH); 4.42 (1H, m, >CH—OH); 5.4 (1H, dd, J=16, ═CH—CH(OH); 6.6 (1H, d, 15.85, Ar—CH═CH); 7.1 (2H, t, Ar—H); 7.6 (2H, dd, Ar—H).

### EXAMPLE 9

### Preparation of Calcium Salt of (2:1)-(+)-7-{4-(4-fluophenyl)-6-isopropyl-2-[methyl(methylsulfonyl)amino]pyrimidin-5-yl}(3R,5S)-3,5-dihyroxy-(E)-hept-6-enoic Acid

To a solution of methyl (6E)-7-{4-(4-flurophenyl)-6-isopropyl-2-[methyl(methyl sulfonyl)amino]pyrimidin-5-yl}(3R,5S)-3,5-dihydroxyhept-6-enoate (2 g, 4.04 mmol) in 30 ml of acetonitrile, 0.25N solution of NaOH (17.7 ml; 4.44 mmol) was added over a period of 5 minutes at temperature between 26-29° C. with stirring. After stirring for 3-4 hours, 30 ml tert-butyl methyl ether was added followed by 10 ml of water. The layers were separated and organic layer was extracted with 20 ml of water. The combined aqueous layers were concentrated by evaporation under reduced pressure to its half volume. To the concentrated aqueous layer, a 1 M solution of CaCl₂.2H₂O (2.02 ml, 2.02 mmol) was added drop wise with stirring at 25-28° C. After stirred for 45 minutes, the precipitate formed was filtered and washed with water to get Rosuvastatin Calcium as a white solid.

### EXAMPLE 10

### Preparation of tert-butyl (4E)-5-{4-(4-flurophenyl)-6-isopropyl-2-[methyl(methylsulfonyl)amino]pyrimidin-5-yl}-3-oxo-4-pentenoate

Diisopropylamine (7.4 g; 73.2 mmol) was taken in 100 ml of dry THF and cooled to −5° C. to 0° C. with stirring under nitrogen atmosphere. To this stirred solution n-butyllithium (1.6M in hexane; 47 ml; 73.2 mmol) was added in drop wise manner over a period of approximately 30 minutes at temperature between −5° C. to +5° C. under nitrogen atmosphere. The reaction mixture was then allowed to reach +10° C. (in the course of 10 minutes) and maintained at that temperature for 30 min. Again the reaction mixture was cooled to around −65° C., tert-butyl acetate (8.5 g; 73.2 mmol) was added in drop wise over a period of 20 minutes and stirred out at that temperature for 40 min. To this, a solution of (2E)-3-{4-(4-flurophenyl)-6-isopropyl-2-[methyl(methylsulfonyl)amino]pyrimidin-5-yl}-2-propenoyl chloride (25.1 g; 61 mmol) in 100 ml of dry THF was added in one lot at −65° C. The reaction mixture was stirred out at temperature between −60° C. and −65° C., the reaction mixture allowed to warm up to −5° C. (in time interval of 45 minutes) and stirred at that temperature for further 30 minutes. The reaction mixture quenched with drop wise addition of acetic acid (50 ml) and stirred for ˜10 minutes. To this 200 ml of ethyl acetate was added followed by 200 ml of water and stirring is carried out for ˜10 minutes. The layers were separated and the aqueous layer was extracted twice with 200 ml of ethyl acetate. The combined organic layers were washed twice with 300 ml saturated NaHCO₃ solution and then with saturated NaCl solution dried over anhydrous Na₂SiO₄, filtered. The filtrate was distilled under reduced pressure to obtained tert-butyl (4E)-5-{4-(4-flurophenyl)-6-isopropyl-2-[methyl(methylsulfonyl)amino]pyrimidin-5-yl}-3-oxo-4-pentenoate as an oily mass.

### EXAMPLE 11

### Preparation of Racemic (4E)-5-{4-(4-flurophenyl)-6-isopropyl-2-[methyl(methylsulfonyl)amino]pyrimidin-5-yl}-3-hydroxy-4-pentenoic Acid

To a stirred solution of tert-butyl (4E)-5-{4-(4-flurophenyl)-6-isopropyl-2-[methyl(methylsulfonyl)amino]pyrimidin-5-yl}-3-hydroxy-4-pentenoate (28.5 g; 57 mmol) in 200 ml of methanol, a solution of aqueous sodium hydroxide (2.54 g; 63.5 mmol in 50 ml water) was added slowly at temperature between 27-29° C. The reaction mixture was heated and refluxed for 6-10 hours. After completion of reaction (completion of reaction was monitored by TLC, ethyl acetate: hexane 3:7), 50 ml of water and 200 ml of tert-butyl methyl ether were added. The organic layer was separated and washed with 100 ml water. The aqueous layers were combined and the pH was adjusted to approximately between 3-4 by acidification and extracted twice with 200 ml of dichloromethane. The combined organic layers were washed with 100 ml saturated NaCl solution, dried over anhydrous Na₂SO₄. The filtrate obtained after filtration was evaporated to dryness under vacuum to obtain racemic (4E)-5-{4-(4-flurophenyl)-6-isopropyl-2-[methyl(methylsulfonyl)amino]pyrimidin-5-yl}-3-hydroxy-4-pentenoic acid as white solid. The structure of the product has been conformed by NMR.

¹H NMR (400 MHz, CDCl₃): 1.2 (6H, d, —CH(CH₃)₃), 2.5 (1H, m, —CH₂—COOH) 3.3 (1H, m, —CH(CH₃)₃), 3.49 (3H, s, —NCH₃), 3.54 (3H, S, —SO₂CH₃), 4.58 (1 h, s, >CH—OH), 5.46 (1H, d, J=15.98, ═CHCOOH), 6.7 (1H, d, J=15.85, —CH═CHCOOH), 7.1 (2H, t, Ar—H), 7.59 (2H, dd, Ar—H). ¹³C NMR (400 MHz, CDCl₃): 21.55, 32.11, 33.10, 40.40, 42.73, 68.09, 114.96, 115.16, 120.86, 124.22, 131.99, 132.08, 134.27, 134.30, 137.32,157.34 161.99, 163.53, 164.47, 174.82, 176.81.

### EXAMPLE 12

### Preparation of Methyl (4E)-5-{4-(4-flurophenyl)-6-isopropyl-2-[methyl(methylsulfonyl)amino]pyrimidin-5-yl}(3S)-3-hydroxy-4-pentenoate

Methanol (25 ml) was taken in a 100 ml three necked round bottomed flask and cooled to −5° C. with stirring. To this acetyl chloride (0.588 g; 7.488 mmol) was added drop wise in such a way that the temperature remains between −5° C. to +5° C. over a period of approximately 10 minutes. After stirring for 30 minutes at 0° C., a solution of (4E)-5-{4-(4-flurophenyl)-6-isopropyl-2-[methyl(methylsulfonyl)amino]pyrimidin-5-yl}(3S)-3-hydroxy-4-pentenoic acid (4.2 g; 9.6 mmol) in 15 ml of methanol was added drop wise over a period of ˜10 minutes at 0° C. and stirred at that temperature for further 30 minutes. Then the reaction mixture was allowed to reach 20-25° C. and stirred for 3-4 hours at 25-29° C. Again the reaction mixture was cooled to 0° C. and 3 g of powered NaHCO₃ was added in portions. The reaction mixture was filtered and to the filtrate 50 ml of ethyl acetate and 30 ml of water were added. The layers were separated and the aqueous layer was extracted twice with 30 ml of ethyl acetate. The combined organic layers were washed with 50 ml of saturated NaHCO₃ solution, 50 ml of saturated NaCl solution and dried over anhydrous Na₂SO₄. Methyl (4E)-5-{4-(4-flurophenyl)-6-isopropyl-2-[methyl (methylsulfonyl)amino]pyrimidin-5-yl}(3 S)-3-hydroxy-4-pententoate was obtained as solid after complete removal of solvent by distillation under vacuum.

¹H NMR (400 MHz, CDCl₃): 1.2 (6H, d, —CH(CH₃)₃), 2.4-2.5 (2H, m, —CH₂COOMe), 3.1 (1H, d, >CH—OH), 3.34-3.41 (1H, m, —CH(CH₃)₃), 3.48 (3H, s, —NCH₃), 3.54 (3H, s, —SO₂CH₃), 3.7 (3H, s, —COOCH₃), 4.6 (1H, s, >CH—OH), 5.5 (1H, dd, J=16.10, 5.12 ═CHCOOCH₃), 6.6 (1H, d, J=16.10, —CH═CHCOOMe), 7.1 (2H, t, Ar—H), 7.6 (2H, dd, Ar—H). ¹³C NMR (409 MHz, CDCl₃): 21.54, 32.03, 33.04, 40.31, 51.85, 68.15, 114.89, 115.10, 121.00, 123.73. 132.00, 132.09, 134.32, 137.71, 157.27, 161.94, 164.42, 172.38, and 174.79.

### EXAMPLE 13

### Preparation of tert-butyl (6E)-7-{4-(4-fluorphenyl)-6-isopropyl-2-[methyl(methylsulfonyl)amino]pyrimidin-5-yl}(5S)-5-hydroxy-3-oxo-6-heptenoate

To a solution of diisopropylamine (0.9 g; 8.87 mmol) in 10 ml of dry tetrahydrofuran, n-butyllithium (1.6M in hexane; 6 ml; 8.87 mmol) was added at 0° C. under nitrogen atmosphere, with stirring in drop wise over a period of ˜10 minutes. The reaction mixture was then allowed to warm up to +10° C. (in time interval of ˜10 minutes) and maintained at that temperature for 30 minutes. Again the reaction mixture was cooled to −65° C. and tert-butyl acetate (1.03 g; 8.87 mmol) was added drop wise over a period of ˜5 minutes. After stirred for another 40 minutes, the resulting solution was transferred to a solution of methyl (4E)-5-{4-(4-flurophenyl)-6-isopropyl-2-[methyl(methylsulfonyl)amino]pyrimidin-5-yl}(3S)-3-hydroxy-4-pentenoate (1 g; 2.2 mmol) in 5 ml of dry THF at 0° C. The reaction mixture was allowed to reach to 20° C. and stirred at that temperature for ˜4 hours. 1 ml of acetic acid was added in drop wise to the reaction mixture followed by 10 ml of ethyl acetate and 10 ml of water. After stirring for 10 minutes, the layers were separated and the aqueous phase was extracted twice with 30 ml of ethyl acetate. The combined organic layers were washed twice with 30 ml saturated NaHCO₃ solution and then with saturated NaCl solution, dried over anhydrous Na₂SO₄. The filtrate obtained after filtration was distilled under vacuum to remove the solvent completely, tert-butyl (6E)-7-{4-(4-flurophenyl)-6-isopropyl-2-[methyl(methylsulfonyl)amino]pyrimidin-5-yl}-(5R)-5-hydroxy-3-oxo-6-heptenoate was obtained as an orange oily mass and was taken as it is for next step.

### EXAMPLE 14

### Preparation of tert-butyl (6E)-7-{4-(4-flurophenyl)-6-isopropyl-2-[methyl(methylsulfonyl)amino]pyrimidin-5-yl}(3R,5S)-3,5-dihydroxyhept-6-enoate

tert-butyl (6E)-7-{4-(4-flurophenyl)-6-isopropyl-2-[methyl(methylsulfonyl)amino]pyrimidin-5-yl}(5S)-5-hydroxy-3-oxo-6-heptenoate (1 g; 1.87 mmol) was taken in 10 ml of dry THF/methanol (4:1 v/v) and cooled to −78°C. under nitrogen atmosphere with stirring. To this stirred solution, diethylmethoxyborane (1 M in THF; 2.1 g; 2.05 mmol)) was added drop wise over a period of ˜5 minutes. After stirring for at that temperature for further 30 minutes, NaBH₄ (0.08 g; 2.05 mmol) was added at −78° C. The reaction mixture was stirred at −78° C. for 3-4 hours. To the reaction mixture 1 ml of acetic acid was added in drop wise followed by 10 ml of ethyl acetate and 10 ml of water. After stirring for 10 minutes at −78° C. the reaction mixture was allowed reach 25-28° C. The layers were separated and the aqueous layer was extracted twice with 30 ml of ethyl acetate. The combined organic phases were washed twice with 30 ml saturated NaHCO₃ solution and then with saturated NaCl solution, dried over anhydrous Na₂SO₄. The reaction mixture was filtered and the solvent was removed by distillation under vacuum. The oily product thus obtained was swapped thrice with 30 ml of methanol to remove borate complex and concentrated to obtain an oily mass, which after column purification provided tert-butyl (6E)-7-{4-(4-flurophenyl)-6-isopropyl-2-[methyl(methylsulfonyl)amino]pyrimidin-5-yl}(3R,5S)-3,5-dihydroxyhept-6-enoate as a solid.

¹H NMR (400 MHz, CDCl₃): 1.23 (6H, d, —CH(CH₃)₃), 1.40-1.50 (1H, m, —C(CH₃)₃, —CH₂), 2.34 (2H, d, —CH₂COO), 3.35 (1H, d, >CH—OH), 3.31-3.38 (1H, m, —CH(CH₃)₃), 3.49 (3H, s, —NCH₃), 3.54 (3H, s, —SO₂CH₃), 3.76 (H, s, —OH), 3.86 (H, s, —OH), 4.41 (1H, d, >CH—OH), 4.42 (1H, t, >CH—OH), 5.42 (1H, dd, J=115.98 ═CHCOO), 6.6 (1H, d, J=16.10, —CH═CHCOO), 7.06 (2H, t, Ar—H), 7.6 (2H, dd, Ar—H).

### EXAMPLE 15

### Preparation of Calcium (2:1)-(+)7-[4-(4-flurophenyl)-6-isopropyl-2[N-methyl-N-methylsulfonylamino)pyrimidin-5-yl]-(3R,5S)-dihydroxy-(E)-6-heptenoic Acid

A solution of tert-butyl (6E)-7-{4-(4-flurophenyl)-6-isopropyl-2-[methyl(methyl sulfonyl)amino]pyrimidin-5-yl}(3R,5S)-3,5-dihydroxyhept-6-enoate (2 g; 3.72 mmol) in 30 ml of acetonitrile of 0.25 M solution of NaOH (14.9 ml; 3.72 mmol) was added over a period of 5 minutes at temperature between 26-29° C. with stirring. After stirred for 3-4 hours, 30 ml of tert-butyl methyl ether was added followed by 10 ml of water. The layers were separated and the organic layer was extracted with 20 ml of water. The combined aqueous layers were concentrated by evaporation under reduced pressure to its half volume. To the concentrated aqueous layer, 1 M solution of CaCl₂.2H₂O (1.86 ml; 1.86 mmol) was added drop wise with stirring at 25-28° C. After stirred for 45 minutes. the precipitate formed was filtered and washed with water to get Rosuvastatin Calcium as a white solid.

1. A process for the manufacture of Rosuvastatin of formula I, according to the present invention, comprising 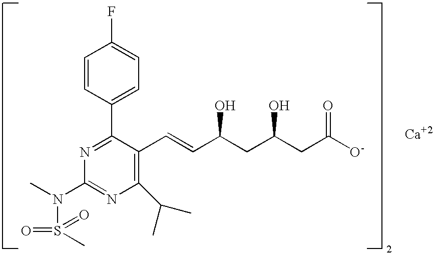
a) reacting a compound of formula (II) 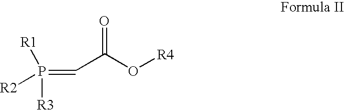
wherein, R1, R2, R3 are substituted or unsubstituted phenyl and R4 is an aliphatic residue selected from C1-C4 alkyl;
with a compound of formula R—CH(═O) (Formula III) wherein R represents the following cyclic structure (formula IV) to obtain a compound of formula (V); 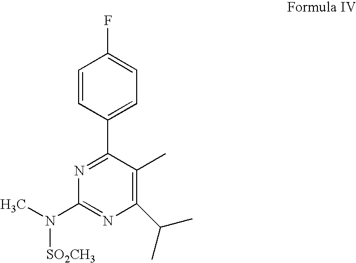
b) hydrolysing a compound of formula (V) to obtain a compound of formula (VI); 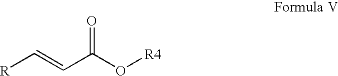
c). treating a compound of formula (VI) 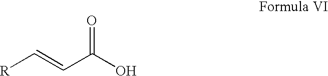
with an acid activating group and subsequently with a compound of formula VII that introduces the radical of formula —CH₂—COOR5 to obtain a compound of formula VIII 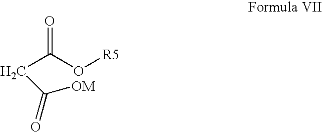
wherein, R5 represents C1-C4 alkyl; M is an alkali metal;
or in another variant of process, converting the compound of formula (VI) to its acid halide of formula (IX) 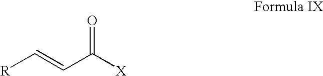
wherein, X represents a halogen
and treating a resulting compound of formula (IX) with a compound of formula (X) to obtain a compound of formula (VIII); 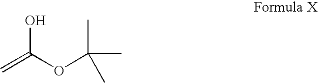
or in another variant of process, treating the compound of formula (IX) with a compound of formula (VII) to obtain a compound of formula (VIII);
d). reducing a compound of formula (VIII) to obtain a compound of formula XI; 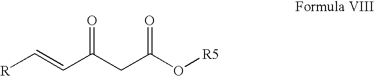
e). hydrolyzing a compound of formula (XI) to obtain a compound of formula XII 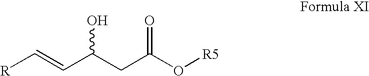
f). resolving the resulting racemic compound of formula (XI), first converting the racemic compound to its diastereomeric salt using the (+) or (−) enantiomeric amine of the formula (XIII) and separating the mixture of diastereomeric salt into the individual diastereomers by chromatography or crystallization and then neutralizing the diastereomeric salt to give the enantiomerically pure product. 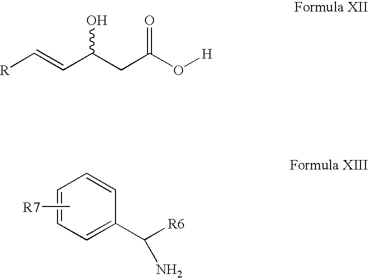
wherein, R6 represent C1-C4-alkyl which is optionally substituted by hydroxyl; R7 represent hydrogen, halogen, C1-C4 alkyl or C1-C4 alkoxy; and
g). treating the resulting compound of formula (XIV) 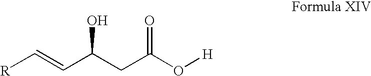
with an acid activating group and subsequently with a compound of formula (VII) that introduces the radical of formula —CH₂—COOR5 to obtain a compound of formula (XV) or in another variant of process, esterifying a compound of formula (XIV) 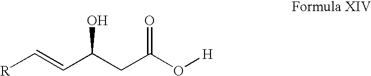
and condensing the resulting compound of formula (XVI) 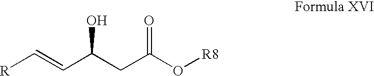
wherein R8 is an aliphatic residue selected from C1-C4 alkyl
with a compound of formula (X) to obtain a compound of formula (XV) 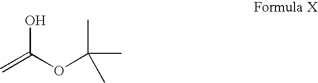
h) reducing a compound of formula (XV) to obtain a compound of formula XVII 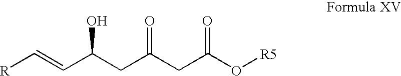
i). hydrolyzing a compound of formula (XVII) and converting, into a salt of formula I thereof 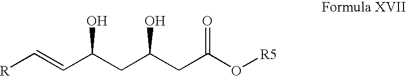
wherein R and R5 have the meanings as defined.

2. A process according to claim 1, wherein the compound of formula II, V, VII, VIII, XI, XV and XVII is used, wherein R4 or R5, respectively, represent C1-C4 alkyl, especially methyl or ethyl or C1-C4 alkyl, especially methyl or ethyl or tert-butyl.

3. A process according to claim 1, wherein the compound of formula XVI is used, wherein R8 represent C1-C4 alkyl, especially methyl or ethyl.

4. A compound of formula VI.

5. A process according to claim 1, the preparation of compound of formula (V) is carried out in a suitable inert solvent, preferably toluene, and in a temperature range from 60° C. to the boiling point of the solvent, preferably at the boiling point of the solvent.

6. A process according to claim 1, the specification of compound of formula (V) is carried out by treating the ester of formula (V) with a strong base, such as an alkali metal hydroxide, preferably NaOH or KOH, in aqueous aliphatic alcohol as solvent, preferably aqueous methanol, and in a temperature range from 25° C. to boiling point of solvent, preferably between 25° C. to 35° C. and acidifying the resulting reaction mixture.

7. A process according to claim 1, formation of compound of formula VIII (step c) is carried out by treating the compound of formula (VI) with an acid activating group. especially preferred one is the use 1,1-carbonyldiimidazole and condensing the resulting compound with alkali metal salt of monoalkylmalonate (formula VII), preferably potassium monomethylmalonate or potassium monoethylmalonate, in presence of magnesium chloride, in an inert organic solvent, preferably tetrahydrofuran, at temperature between 0-40° C., preferably at 0-35° C.

8. A process according to claim 1, in another variant of process to prepare compound of formula VIII is carried out by first converting compound of formula VI to a compound of formula (IX) in an inert solvent, preferably dichloromethane, and in temperature range from 0° C. to boiling point of the solvent, preferably between 0° C. to 28° C. using oxalyl chloride or thionyl chloride and subsequent treatment of a resulting compound of formula (IX) with compound of formula (X) in the presence of a suitable base and in a suitable inert solvent, especially tetrahydrofuran, and in a temperature range from −78° C., to the boiling point of the solvent, preferably at −78 to room temperature.
A suitable base is selected from an alkane alkali metal in presence of diisopropylamine, alkali alkylsilazanes like LiHMDS or NaHMDS. Especially preferred is the use of n-butyllithium in the presence of diisopropylamine.

9. A process according to claim 1, in another variant of process to prepare compound of formula VIII is carried out by condensing a compound of formula (IX) with an alkali metal salt of monoalkylmalonate (formula VII), preferably potassium mononethylmalonate or potassium monoethylmalonate, in presence of magnesium chloride, in an inert organic solvent, preferably tetrahydrofuran, at temperature between 0-40° C., preferably at 0-35° C.

10. A process according to claim 1, reduction of compound of formula VIII (step d), is carried out in a mixture of an inert solvent, such as an ether, preferably tetrahydrofuran and a lower alkanol, preferably methanol, in the ratio of 4:1 volume/volume, and at temperature range from −78° C. to 0° C., preferably at −65° C. to 0° C.
A preferred reduction agent is a hydride, for example, an alkalimetal borohydride, especially sodium borohydride.

11. A process according to claim 1, saponification of compound of formula XI (step e) is carried out by treating the ester of formula (XI) with a strong base, such as an alkali metal hydroxide, preferably NaOH or KOH, in aqueous aliphatic alcohol as solvent, preferably aqueous methanol, and in a temperature range from 25° C. to boiling point of solvent, preferably between 25° C. to 30° C. and acidifying the resulting reaction mixture.

12. A process according to claim 1, resolution of racemate of compound of formula XII (step f), in to optically pure antipodes is carried out by means of preparative chromatography using chiral supports (HPLC) or by crystallization using optically pure precipitating agents, for example (+) or (−) phenylalkylamine or substituted phenylalkylamine, preferably (R)-1-phenylethylamine in alcoholic solvents such as lower alkanol, preferably ethanol and recrystallising from a mixture of ketonic solvent and lower alkanol, preferably mixture of acetone and methanol followed by neutralization.

13. A process according to claim 1, formation of compound of formula XV (step g) is carried out by treating the compound of formula XIV with an acid activating group, especially preferred one is the use of 1,1-carbonyldiimidazole and condensing the resulting compound with an alkali metal salt of manoalkyl malonate (formula VII), preferably potassium monomethylmalonate or potassium monoethylmalonate, in presence of magnesium chloride, in an inert solvent, preferred one is tetrahydrofuran, at temperature between 0-40° C., preferably at 0-35° C.

14. A process according to claim 1, in another variant of process to prepare compound of formula XV is carried out by converting compound of formula XIV to a compound of formula XVI by esterification and condensing the resulting compound of formula XVI with a compound of formula X.
Esterification of compound of formula XIV is carried out, in lower alcoholic solvent, especially C1-C3 alkanol, preferably methanol, in presence of acid catalyst like inorganic acids or p-toluensulphonic acid or acidic resins, and in a temperature range from 0° C. to boiling point of solvent, preferably between 0° C. to 30° C.
Condensation step is carried out in the presence of a suitable base and in a suitable inert solvent, especially tetrahydrofuran, and in a temperature range from 78° C. to the boiling point of the solvent, preferably at room temperature. A suitable base is selected from an alkane alkalimetal in the presence of diisopropylamine, alkali alkylsilazanes like LiHMDS or NaHMDS. Preferred one is the use of n-butyllithium in the presence of diisopropylamine.

15. A process according to claim 1, reduction of compound of formula XV (step h), is carried out in a mixture of an inert solvent, preferably tetrahydrofuran and lower alkanol, preferably methanol, in the ratio of 4:1 volume/volume, and at temperature from −78° C. to 0° C., preferably at −78° C. to −70° C. To split the corresponding boronic ester the reaction mixture is then treated with methanol, and in a temperature range from 0° C. to the boiling point of solvent, preferably in range of 0° C. to 40° C.
A preferred reduction agent is an alkali metal borohydride in the presence of a di-C1-C7-alkyl-C1-C4 alkoxy-borane, preferably sodium borohydride in presence of diethylmethoxyborane.

16. A process according to claim 1, formation of compound of formula I (step i), is carried out first saponification of compound of formula XVII using a base, such as an alkali metal hydroxide, preferably NaOH followed by treatment with aqueous calcium chloride solution.
